(19) United States
(12) Patent Application Publication
(10) Pub. No.: US 2021/0284982 A1
(43) Pub. Date: Sep. 16, 2021

(54) METHOD FOR THE PRODUCTION OF CELLULASES USING A FILAMENTOUS FUNGUS

(71) Applicant: IFP Energies Nouvelles, Rueil-Malmaison (FR)

(72) Inventors: Mohamed Fadhel BEN CHAABANE, Rueil-Malmaison (FR); Celine COHEN, Rueil-Malmaison (FR); Bernard CHAUSSEPIED, Rueil-Malmaison (FR); Frederic AUGIER, Rueil-Malmaison (FR)

(73) Assignee: IFP Energies Nouvelles, Rueil-Malmaison (FR)

(21) Appl. No.: 17/275,703

(22) PCT Filed: Aug. 26, 2019
(86) PCT No.: PCT/EP2019/072722

§ 371 Date: Mar. 12, 2021

(30) Foreign Application Priority Data
Sep. 14, 2018 (FR) 1858322

(51) Int. Cl. C12N 9/42; C12P 7/10

(52) U.S. Cl. CPC C12N 9/2437; C12P 7/10

(57) ABSTRACT

The present invention relates to a method for producing enzymes by a strain belonging to a filamentous fungus, which comprises three steps:
(a) a first step of growing biomass, in the presence of at least one carbon-based growth substrate in a stirred and aerated bioreactor (1) in batch phase,
(b) a second step of diluting the culture medium obtained in the first step (a),
(c) a third step of producing enzymes from the diluted culture medium obtained in the second step (b), in the presence of at least one inductive carbon-based substrate, in fed-batch phase.

### FIELD OF THE INVENTION

The invention relates to a method for producing enzymes by a filamentous fungus, especially enzymes of the cellulase (cellulolytic and/or hemicellulolytic) type, which are needed for the enzymatic hydrolysis of lignocellulosic biomass.

This enzymatic hydrolysis is employed, for example, in “second generation” (2G) processes for producing biofuels such as bioethanol, or for producing sugary juices which can be used for producing other products via a chemical or biochemical/fermentation pathway (for example, alcohols such as ethanol, butanol, propanol, isopropanol, or other molecules, for example, solvents such as acetone, and other biobased molecules), or in other processes in the chemical, paper or textile industries.

### PRIOR ART

Trichoderma reesei is the microorganism most widely used in the production of cellulases on an industrial scale. The wild-type strains have the faculty, in the presence of an inductive substrate, cellulose for example, of excreting an enzymatic complex which is considered to be well suited to the hydrolysis of lignocellulosic biomass. The enzymes in the enzymatic complex contain three major types according to their activity: endoglucanases, exoglucanases, and cellobiases. Other proteins possessing properties vital for the hydrolysis of lignocellulosic materials are also produced by Trichoderma reesei, xylanases for example.

The presence of an inductive substrate is essential for the expression of the cellulolytic and/or hemicellulolytic enzymes. The nature of the carbon-based substrate has a strong influence on the composition of the enzymatic complex. This is the case for xylose, which, when combined with a carbon-based inductive substrate such as cellulose or lactose, produces a significant improvement in the activity referred to as xylanase. Regulation of the cellulase genes has been studied in detail on a variety of carbon sources. They are induced in the presence of cellulose, of its hydrolysis products (for example: cellobiose), or of certain oligosaccharides such as lactose or sophorose (Ilmén et al., 1997; Appl. Environ. Microbiol. 63: 1298-1306).

Conventional genetic mutation techniques have enabled the selection of strains of Trichoderma reesei which hyperproduce cellulases, such as the strains MCG77 (Gallo—U.S. Pat. No. 4,275,167), MCG 80 (Allen, A. L. and Andreotti, R. E., Biotechnol.-Bioeng. 1982, 12, 451-459 1982), RUT C30 (Montenecourt, B. S. and Eveleigh, D. E., Appl. Environ. Microbiol. 1977, 34, 777-782) and CL847 (Durand et al., 1984, Proc. Colloque SFM “Génétique des microorganismes industriels [Genetics of industrial microorganisms]”. Paris. H. Heslot Ed, pages 39-50).

The process for producing cellulases by Trichoderma reesei has been a subject of substantial development studies for extrapolation to the industrial scale. To obtain high enzyme productivities, it is necessary to supply a source of rapidly assimilable carbon for the growth of Trichoderma reesei, and an inductive substrate which allows the expression of the cellulases and secretion into the culture medium. Cellulose is able to fulfill these two functions. However, cellulose is difficult to use on an industrial scale, since it increases the viscosity of the medium and may disrupt the transfer of oxygen, which is vital to the growth of the microorganism, which is strictly aerobic. It can be replaced by soluble carbon sources, such as glucose, xylose or lactose, with lactose also acting as an inductive substrate. Other soluble sugars such as cellobiose and sophorose have been described as inductive, but they may be considered to be too expensive for use on an industrial stage.

It has been found that production of cellulases by Trichoderma reesei with soluble substrates is very much inferior to that obtained on “batch” cellulose. This is because of the repressive effect of the readily assimilable sugars at high concentration. Continuous feeding of soluble carbon-based substrates in fed-batch mode has made it possible to raise the catabolic repression by limiting the residual concentration in the cultures and by optimizing the amount of sugar, making it possible to obtain a better yield and better enzymatic productivity, as described in patent FR-B-2 555 603. This protocol results in a protein concentration of the order of 35 to 40 g/L with a productivity of the order of 0.2 g/L/h.

To achieve this performance, the industrial processes for producing cellulases, as are described in the abovementioned patent, thus take place in two steps in the same reactor:

A growth step in “batch” mode, where it is necessary to supply a source of rapidly assimilable carbon for the growth of Trichoderma reesei. This phase is characterized by an increase in the viscosity of the medium, and by a high oxygen demand.

A step of production in “fed-batch” mode, using an inductive substrate (lactose, for example) which allows expression of the cellulases and secretion into the culture medium. The optimum flow applied is between 35 and 45 mg (of sugar)·g (of dry cellular weight)⁻¹·h⁻¹. This phase is characterized by a drop in the viscosity of the medium and by a lower oxygen demand.

The protein productivity is equal to the product of the concentration of living biomass and the specific rate of protein production. This productivity may be enhanced by increasing the performance of production of the strain (increasing the specific rate) and/or by increasing the concentration of cellular biomass. However, methods for improving the performance of these strains, by subjecting them to genetic modification as set out earlier on above, may reach a limit, and increasing the concentration of biomass results in a sharp increase in the viscosity, thereby limiting the transfer of oxygen, in particular at the end of the growth step.

The production steps proposed in the prior art take place in stirred tanks, with the steps of growth and of production taking place in the same bioreactor. This is a solution of interest, being compact in terms of industrial plant, but it is not necessarily the best solution, since the two steps, of growth and of production, do not have the same requirements, in terms, for example, of stirring means or of oxygen transfer: the bioreactor must therefore have the volume and all of the equipment and operating means that allow it to ensure these two steps together, although said steps proceed under quite different conditions.

It is therefore the aim of the invention to propose an improved method for producing cellulasic enzymes from filamentous fungal strains, especially a method which enables increased enzyme productivities and, secondarily, which can be implemented on an industrial scale in plants which enable more flexibility.

### SUMMARY OF THE INVENTION

The present invention concerns a method for producing enzymes by a strain belonging to a filamentous fungus, said method comprising the three following steps:

(a) a first step of growing the fungi, in the presence of at least one carbon-based growth substrate, in a stirred and aerated bioreactor in batch phase,

(b) a second step of diluting (in volume terms) the culture medium obtained in the first step (a),

(c) a third step of producing enzymes from the diluted culture medium obtained in the second step (b), in the presence of at least one inductive carbon-based substrate, in fed-batch phase.

The invention includes a method of this kind where step (b) may commence (a little) before the end of step (a) or may finish (a little) after the start of step (c). The invention therefore includes a method of this kind with an implementation in which steps (a) and (b) on the one hand and steps (b) and (c) on the other may partially overlap one another. The reason, as detailed later on below, is that the dilution step (b) may last several hours, and it may therefore be advantageous to commence it between 0 and 4 hours before the end of step (a), and/or to finish it between 0 and 4 hours after the transition to step (c).

The invention has therefore developed a new fermentation regime, with an intermediate dilution step between the growth step and the production step. Entirely surprisingly, it has emerged that the specific productivities obtained after dilution of the culture medium at the end of the growth phase are significantly higher than those obtained with the culture medium without dilution (the increase may be as much as 30% or more), for the same specific flow of carbon-based substrate (calculated relative to the mass of biomass in the reactors).

The third production step (c) is preferably carried out in a bubble column: In this preferred embodiment, the invention also judiciously exploits the idea that the growth step and production step do not have the same requirements and do not have the same procedure, particularly in terms of stirring and of oxygen transfer, and that it is therefore more relevant to carry out the steps in two different fermenters: The production of biomass may be performed under good conditions in a stirred tank, with the objective preferably of a high concentration of biomass (of greater than or equal to 20 g/L, for example). Production is performed in a bubble column, this being a particularly appropriate reactor for performing production, since the reactor is suitable for dilute media of low viscosity, and also has many advantages, such as being very easy to clean, having a greater energy efficiency than stirred bioreactors, and allowing high reaction volumes to be offered. (In the present text, “stirred bioreactor” has the same meaning as “stirred tank”).

The second step (b) of dilution is carried out advantageously in the bioreactor at the end of the fungi growing step (a) and/or in the bubble column before or at the start of the third step (c) of producing enzymes.

The step of dilution, which therefore leads to a drop in the viscosity of the culture medium, may thus be carried out in either of the reactors, or even in line during the transfer of the contents of the first reactor into the second reactor. Preference may be given to performing the dilution in the stirred tank, so as to make it easier to transfer the already diluted medium from the stirred tank to the bubble column.

In the dilution step (b), the factor by which the culture medium is diluted is advantageously at least one 1.1 or 1.2, in particular at least 1.5, preferably approximately 2, and in particular at most 6. Dilution is to be adjusted so that the culture medium has a biomass concentration which allows the viscosity of the medium to be limited—that is, a concentration of between 5 and 20 g/L. In the present invention, the term biomass corresponds to the fungi.

The dilution factor is a volume dilution factor, and corresponds to the ratio of the volumes after dilution and before dilution. Since the culture medium is an aqueous medium, the dilution liquid is preferably water or an aqueous medium, such as a culture medium as well.

According to one preferred embodiment, means are provided for fluidic connection between the bioreactor and the bubble column to ensure the transfer of the culture medium obtained in the growth step in the bioreactor to the bubble column, said means taking the form, in particular, of pipes equipped with manual or controlled valves and in particular with pump(s). In this scenario, the second step (b) of dilution may be carried out wholly or partly in the fluidic connection means during the transfer of the culture medium by these fluidic connection means from the bioreactor to the bubble column.

The concentration of carbon-based growth substrate in the bioreactor is preferably greater than 20 g/L, in particular between 30 and 100 g/L, and preferably between 50 and 80 g/L.

The inductive carbon-based substrate preferably feeds the bubble column at a specific rate of between 30 and 140 mg per gram of cellular biomass per hour, preferably between 35 and 45 mg per gram of cellular biomass per hour.

The step of growing the biomass is advantageously continued up to a biomass (fungi) concentration of at least 20 g/L. The product is therefore a concentrated culture medium, which will be subsequently diluted before production is commenced.

According to one embodiment, it is possible to implement the method of the invention with a number n of stirred and aerated bioreactors and a number m of bubble columns, where n is less than or equal to m, it being possible for one bubble column to be fluidically connected to two or more bioreactors or vice versa. It is possible to have bubble columns with a much greater capacity than stirred bioreactors, thereby enabling two or more stirred bioreactors to feed a single bubble column, for example, and to take account of the fact that the growth step is quicker than the production step.

The carbon-based growth substrate is preferably selected from at least one of the following compounds: lactose, glucose, xylose, the residues obtained after ethanolic fermentation of the monomeric sugars from the enzymatic hydrolysates of cellulosic biomass, and a crude extract of water-soluble pentoses from the pretreatment of a cellulosic biomass.

The inductive carbon-based substrate is preferably selected from at least one of the following compounds: lactose, cellobiose, sophorose, the residues obtained after ethanolic fermentation of the monomeric sugars from enzymatic hydrolysates of cellulosic biomass, and a crude extract of water-soluble pentoses from the pretreatment of a cellulosic biomass.

The aforesaid inductive and growth substrates may be used alone or as a mixture.

Depending on its nature, the carbon-based growth substrate selected for obtaining the biomass is introduced into the bioreactor before sterilization, or is sterilized separately and introduced into the bioreactor after sterilization.

The inductive carbon-based substrate introduced during the fed-batch production step is preferably sterilized independently, before being introduced into the bubble column. If the inductive carbon-based substrate is the pretreated biomass or hemicellulosic hydrolysates, it is possible to use it without sterilization.

Preferably, when the inductive carbon source is lactose, the aqueous solution is prepared at a concentration of 200-250 g·L⁻¹. If the inductive carbon-based substrate is pretreated biomass, the aim is preferably for a mean supply of sugar of between 35 and 140 mg per gram of cell per hour.

The strains used preferably belong to the species Trichoderma reesei, optionally modified for enhancing the cellulolytic and/or hemicellulolytic enzymes by methods of mutation-selection; the strains enhanced by techniques of generic recombination may also be used. These strains are cultured under conditions that are compatible with their growth and the production of the enzymes. Other microorganism strains producing enzymes by similar processes to those used for Trichoderma may be employed.*

The enzymes are preferably cellulolytic or hemicellulolytic enzymes.

The filamentous fungal strain used is preferably, therefore, a strain of Trichoderma reesei or of Trichoderma reesei modified by mutation, selection or genetic recombination. Very preferably the strain is a CL847, RutC30, MCG77 or MCG80 strain.

A further subject of the invention is a plant for producing enzymes by a strain belonging to a filamentous fungus, such that said plant comprises:—a stirred and aerated bioreactor operating in batch phase for performing a step of growing the fungi, in the presence of at least one carbon-based growth substrate;—a bubble column operating in fed-batch phase for performing the step of producing enzymes from the culture medium from the bioreactor; —means of fluidic connection connecting the bioreactor to the bubble column for performing the transfer of the growth medium from the bioreactor to the bubble column;—means for injecting the dilution liquid into the bioreactor and/or into the bubble column and/or into the means of fluidic connection. The bioreactor and/or the bubble column preferably contain a culture medium with a strain belonging to a filamentous fungus.

This plant may advantageously implement the method described above, with the same resulting advantages: better specific productivity than a fermentation without intermediate dilution, in a single stirred tank, easier performance and maintenance of the plant, adaptation to productions of variable amounts, etc.

The bubble column advantageously has an internal volume at least two times greater than the internal volume of the stirred bioreactor, thereby allowing the bubble column to “absorb” the increased volume of the diluted culture medium and/or allowing it to be fed by two or more stirred bioreactors.

The plant described above may therefore comprise n stirred and aerated bioreactors and m bubble columns, where n is less than or equal to m (and where n is greater than or equal to 1).

### DESCRIPTION OF THE FIGURES

FIG. 1 is a highly schematic representation of the plant implementing the method of the invention.

FIGS. 2a, 2b and 2c are graphs showing the change in concentration of the mass of the proteins produced and of the mass of the cellular biomass according to three different examples.

### DETAILED DESCRIPTION OF THE INVENTION

In one preferred embodiment, the present invention relates to a method for producing cellulases by a strain belonging to the species Trichoderma reesei, in a stirred and aerated bioreactor, comprising three steps:

the first step of growing in the presence of at least one carbon-based growth substrate in batch phase, with a concentration of carbon-based growth substrate of between 30 and 100 g/L, preferably from 50 to 80 g/L. This step takes place in a stirred tank.

a second step of diluting the fermentation must by a factor of more than 1.1 and preferably of 2. This dilution step may be carried out in the growth tank. Another solution involves carrying out dilution in line during the transfer of the contents of the stirred tank to the bubble column.

a third step of production in a bubble column, in the presence of at least one inductive carbon-based substrate in fed-batch phase, this substrate being fed at a specific rate of between 35 and 140 mg per gram of cells per hour, and preferably between 35 and 45 mg per gram of cells per hour.

The production step is carried out under conditions of limitation of the inductive carbon-based substrate, with a flow less than the maximum capacity of consumption of the strain.

Various carbon-based inductive and growth substrates have already been listed earlier on above. Preferably, when the inductive carbon source is lactose, the aqueous solution is prepared at a concentration of 200-250 g·L⁻¹. When the inductive carbon-based substrate is pretreated biomass, the aim is for an average supply of sugar of between 35 and 140 mg per gram of cell per hour.

The method according to the present invention enables superior cellulase productivity by making better use of stirred bioreactors: dedicated to the growth of biomass, they can be arranged and adapted more effectively to this step only. The biomass is subsequently transferred into bubble columns, which are large-volume fermenters and which themselves would be dedicated to the production of enzymes. The method in its entirety is more economical in terms of utilities (energy consumption, etc.) and more flexible.

The regime has therefore been adapted relative to the conventional processes, firstly by diluting the fungal concentration at the end of growth, and secondly by carrying out production in a bubble column.

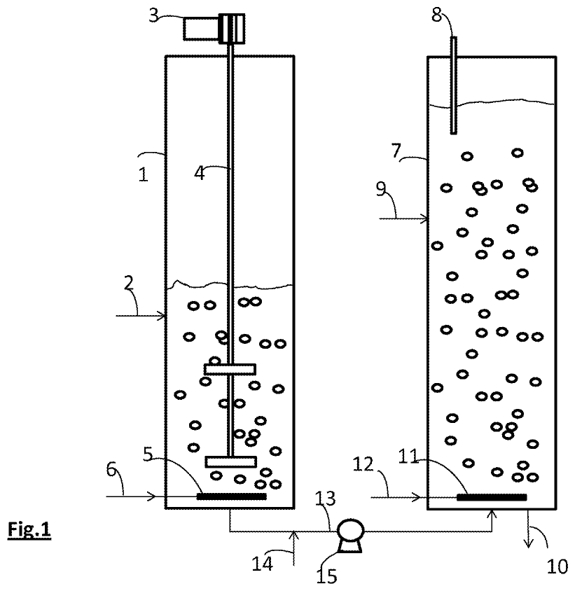

An exemplary embodiment of the method according to the invention is shown in FIG. 1, which represents one example of a plant implementing the method.

The references have the following meaning:

1: stirred tank

2: feed of the stirred tank

3: motor

4: stirring device

5: bubbling device

6: air feed

7: bubble column

8: feed of substrate

9: feed of the bubble column

10: discharge point

11: bubbling device

12: air feed

13: transfer line

14: injection point

15: pump

The plant therefore comprises, first of all, a stirred tank 1, in which the growth step will be carried out. This tank 1 has a volume of between 20 and 500 m³, preferably of between 40 and 400 m³.

The stirred tank 1 comprises between one and five rotary stirrers 4, with a stirring power of between 0.1 and 7 kW/m³, with preferably a maximum power of between 1 and 4 kW/m³ by the motor 3. The tank 1 has a height/diameter ratio of between 1 and 5, preferably of between 1.5 and 3. The tank 1 is also equipped with a bubbling device 5 in the lower part, fluidically connected to an external air intake 6, and with one (or more) intakes 2 for carbon-based growth source and for fungi.

The tank 1 operates at between 1 and 5 bar abs. It is fed with air (5,6) at a rate of between 0.1 and 2 volumes of air (under standard conditions) per volume of liquid per hour, preferably between 0.2 and 1 volume of air per volume of liquid per hour.

The plant further comprises a bubble column 7, to carrying out the production step. As a reminder, a bubble column is a reaction chamber composed of a cylindrical column, with a height-to-diameter ratio conventionally between 1 and 10, preferably between 2.5 and 6. The column is equipped with an air injection device, optionally enriched in oxygen. This injection device is positioned at the bottom of the column, so that the air injected feeds the entirety of the whole of the volume with bubbles and therefore with oxygen. The injection device may be a perforated plate, a system of perforated tubes, or any other system known to a person skilled in the art. The application “Bubble Column Reactors” by W. D. Deckwer (J.Wiley & Sons, 1992) provides a comprehensive description of bubble columns, which may take multiple variant forms, depending on whether they are empty or equipped with perforated plate internals, recirculation pipes or other components.

The simplified representation of the bubble column 7 according to FIG. 1 represents the feeding 8 of the bubble column with inductive carbon-based substrate, a bubbling device 11 in the lower part in fluidic connection with an external air intake 12.

The bubble column has a volume of between 40 and 1000 m³, and in general defines an interior volume at least two times larger than that of the stirred tank 1, to allow the dilution of the culture medium obtained from the growth in tank 1. The bubble column 7 has a height/diameter ratio of between 2 and 10, preferably between 2.5 and 7.

As set out earlier on above, the bubble column may be empty, or may be equipped with an internal cylinder to promote the recirculation of the liquid. A person skilled in the art then refers to the reactor as an “airlift” reactor, which is a well-known variant of the simple bubble column and which may also be used in the context of the present invention.

Like the tank 1, the bubble column 7 operates at between 1 and 5 bar abs. It is fed with air 11,12 at a flow rate of between 0.1 and 2 volumes of air (under standard conditions) per volume of liquid per hour, preferably between 0.2 and 1 volume of air per volume of liquid per hour.

The culture medium is transferred from the stirred tank 1 to the bubble column 7 via a fluidic connection 13 between the two reactors, in the form of one or more pipes connecting the two reactors. In this case, these pipes are disposed in bottom parts of the two reactors, both being essentially oriented according to a vertical axis. These pipes are equipped with a pump 15, and with manual or controlled valves which allow the transfer of the medium from the tank 1 to the bubble column 7 to be executed.

The step of diluting the culture medium between the growth step in the tank 1 and the production step in the bubble column may be carried out in three different ways:—alternatively in the tank 1, at the end of growth, by supplying aqueous liquid to the injection point 2, before transfer to the column 7—or at the fluidic connection, by supplying aqueous liquid to the culture medium in the course of transfer (injection point 14),—or at the entry of the column 7, by supplying aqueous liquid to the column 7 at the injection point 9. A choice may also be made to mix these three modes of dilution, in other words to add some of the required water in the tank and a further portion during transfer and/or in the bubble column, for example. The goal is to reach the required level of dilution, of approximately 2, for example, depending on the respective volumes of the two reactors, on the viscosity of the culture medium at the end of the growth step, etc.

The prior dilution step according to the invention therefore makes it possible in particular to adjust the final concentration of biomass to a target concentration of between 5 and 20 g/L.

Given the different times for growth and for production (a factor of 3 to 8 may exist between the times of the two steps, with growth always being the shorter step), the industrial enzyme production method according to the invention is able to use a smaller number of stirred tanks than the number of bubble columns, thereby making it possible to maximize the use of the two types of fermenters.

For example, if step (a) lasts 50 hours and step (c) lasts 200 hours, the contents of the stirred tank may be transferred, after dilution (b), into a first bubble column, and then the tank may be reused for carrying out step (a) once again. The new contents of the tank will then be diluted and transferred to another bubble column. The number of bubble columns and of stirred reactors is to be adjusted as a function of the duration of each step.

### EXAMPLES

Example 1 is an example comparing two productions stages carried out at respective concentrations of 12.5 g/L and 25 g/L of stabilized biomass during the fed-batch production step. Example 2 according to the invention demonstrates the feasibility and the production performance obtained with a regime carried out in the stirred tank during the growth step and in a bubble column during the enzyme production step.

Comparative example 3 demonstrates the difficulty of carrying out the growth step in a bubble column without observing the conditions of the invention.

### Example 1 (Comparison—Preliminary to the Invention)

Cellulases are produced in a mechanically stirred fermenter (not shown in FIG. 1). The mineral medium has the following composition: KOH 1.66 g/L, H3PO4 85% 2 mL/L, (NH₄)2SO₄ 2.8 g/L, MgSO₄, 7 H₂O 0.6 g/L, CaCl₂ 0.6 g/L, MnSO₄ 3.2 mg/L, ZnSO_{4,} 7_{H2O} 2.8 mg/L, CoCl₂ 10 4.0 mg/L, FeSO₄, 7 H₂O 10 mg/L, corn steep 1.2 g/L, antifoam 0.5 mL/L.

The fermenter containing the mineral medium is sterilized at 120° C. for 20 minutes; the carbon-based glucose source is sterilized separately at 120° C. for 20 minutes and then added in a sterile manner to the tank 1 so as to have a final concentration of 25 g/L. The fermenter is seeded at 10% (v/v) with a liquid preculture of the Trichoderma reesei strain CL847. The mineral medium of the preculture is identical to that of the fermenter, except for the addition of potassium phthalate at 5 g·L⁻¹ to buffer the pH of the medium. The growth of the fungus in preculture is performed using glucose as carbon-based substrate, at a concentration of 30 g·L⁻¹. The growth of the inoculum lasts 2 to 3 days, and is performed at 28° C. in a shaking incubator. Transfer to the fermenter is performed when the residual glucose concentration is less than 15 g/L.

The growth step is performed for 50 hours in the stirred bioreactor 1 with an initial concentration of 50 L of glucose, at a temperature of 27° C. and a pH of 4.8 (adjusted with 5.5 M aqueous ammonia). The aeration is 0.5 vvm (volume/volume/minute), and the partial pressure of oxygen dissolved in the medium is 40% of P 02sat. After exhaustion of the substrate, the 50 g/L of glucose produced a biomass concentration of approximately 25 g/L. A portion of the medium is withdrawn and diluted by a factor of two in another sterile bioreactor identical with the one before it, so as to have, at the start of the fed-batch production phase, one bioreactor containing 25 g/L of biomass and another containing 12.5 g/L of biomass.

A 250 g/L lactose solution is injected continuously at a rate of 35 to 45 mg per g of cells per hour for 164 hours into the two reactors (approximately 2 mL/h for the experiment with 12.5 g/L of biomass and 4 mL/h for the experiment with 25 g/L of biomass). The temperature is lowered to 25° C., and the pH is maintained at 4 until the end of culturing. The pH is adjusted by adding a 5.5 N aqueous ammonia solution, which provides the nitrogen needed for the synthesis of the excreted proteins. The dissolved oxygen content is maintained at 40% of P O₂sat.

The production of enzymes is monitored by assaying the extracellular proteins using the Lowry method and BSA standard, after separation of the mycelium by filtration or centrifuging.

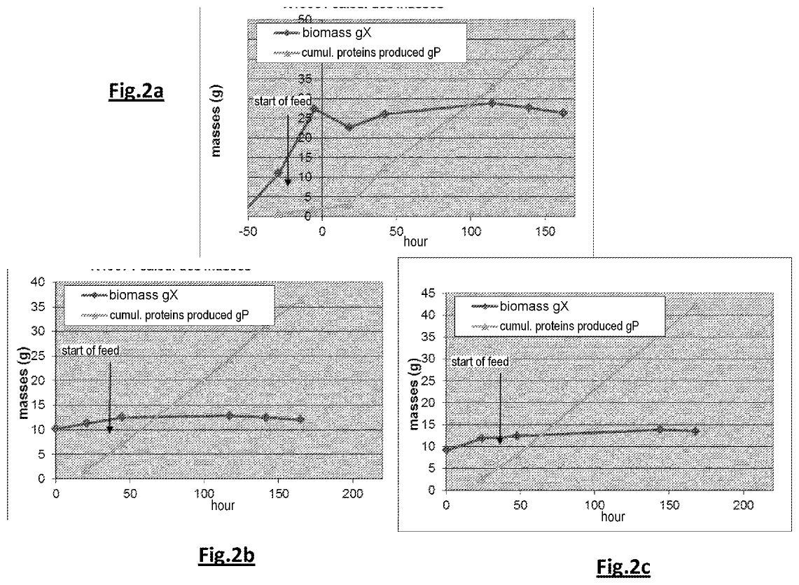

FIG. 2a shows the change in the mass of cellular biomass and of proteins for the experiment with 25 g/L of biomass, and FIG. 2b shows the same curves but for a stabilized biomass at 12.5 g/L. A comparison of the two graphs shows that 47 g of proteins are obtained with the experiment with 25 g/L of biomass, and only 36 g of proteins with the experiment at 12.5 g/L of biomass. In contrast, the specific rate of protein production, identified as gp, is markedly greater with the experiment at 12.5 g/L of biomass (approximately 65% greater). It is in fact 12±2 mg/g/h at 25 g/L of biomass and 20±1 mg/g/h with the experiment at 12.5 g/L of biomass.

This comparison shows that, surprisingly, a specific rate which is at least 50% higher is obtained with a biomass concentration of 12.5 g/L, relative to a concentration of 25 g/L. This shows the advantage of carrying out dilution prior to the production step, with this determining the mode of production presented in inventive example 2 below.

### Example 2 (According to the Invention)

The experiment is carried out under the same conditions as example 1, except that after the step of dilution (for transition from a concentration of 25 to 12.5 g/L of cellular biomass) in the stirred tank 1, the production step is carried out in the bubble column 7, after transfer from one reactor to the other via the piping system 13, with stirring therefore taking place in production solely by injection of air at a vvm of 1 into the bubble column.

The changes in the masses of the cellular biomass and in the protein mass are represented in FIG. 2c: It is seen from this graph that the final mass of proteins obtained is 42 g (after 168 h of fed-batch production). The calculated specific rate of protein production is 22±1 mg/g/h.

### Example 3 (Comparative)

Example 3 is carried out under the same conditions as example 1, but with simulation of a growth step in a bubble column, as in production: In this example, growth is carried out in a stirred tank but without stirring, to imitate operation in a bubble column.

The result of this was a culturing failure. It was not possible to measure the concentration of biomass, which formed a large ball around the stirring shaft, which had not been activated.

In conclusion, the invention integrates an intermediate dilution step with very advantageous and unexpected effects on the performance of the method. It proved very advantageous, furthermore, to use two different types of reactor to perform the growth step and production step, so that these steps take place under industrially optimal conditions: the invention allows the transition of protein production to the industrial scale in a manner which is robust and effective, remaining simple and very flexible in its implementation.

1. A method for producing enzymes by a strain belonging to a filamentous fungus, characterized in that said method comprises three steps:
(a) a first step of growing the fungi, in the presence of at least one carbon-based growth substrate, in a stirred and aerated bioreactor in batch phase,
(b) a second step of diluting the culture medium obtained in the first step (a),
(c) a third step of producing enzymes from the diluted culture medium obtained in the second step (b), in the presence of at least one inductive carbon-based substrate, in fed-batch phase.

2. The method as claimed in claim 1, wherein the third step (c) of producing enzymes is carried out in a bubble column (7).

3. The method as claimed in claim 1, wherein in the dilution step (b) the culture medium is diluted by a volume factor selected from at least 1.1 at least 1.2, at least 1.5, approximately 2, and at most 6, so as to attain a fungal concentration of between 5 and 20 g/L.

4. The method as claimed in claim 1, wherein the second step (b) of dilution is carried out in the bioreactor, at the end of the growth step (a) and/or in the bubble column before or at the start of the third step (c) of producing enzymes.

5. The method as claimed in claim 1, wherein means (13) are provided for fluidic connection between the bioreactor and the bubble column to ensure the transfer of the culture medium obtained in the growth step in the bioreactor to the bubble column, said means taking the form, in particular, of pipes equipped with manual or controlled valves and in particular with pump(s).

6. The method as claimed in claim 1, wherein the second step (b) of diluting is carried out wholly or partly in the means (13) of fluidic connection during the transfer of the culture medium via said means of fluidic connection from the bioreactor to the bubble column.

7. The method as claimed in claim 1, wherein the concentration of carbon-based growth substrate in the bioreactor is between 30 and 100 g/L, including between 50 and 80 g/L.

8. The method as claimed in claim 1, wherein the inductive carbon-based substrate feeds the bubble column at a specific rate of between 30 and 140 mg per gram of cellular biomass per hour, including between 35 and 45 mg per gram of cellular biomass per hour.

9. The method as claimed in claim 1, wherein the growth step is continued to a fungal concentration of at least 20 g/L.

10. The method as claimed in claim 1, which is performed with a number n of stirred and aerated bioreactors and a number m of bubble columns, where n is less than or equal to m, it being possible for one bubble column to be connected fluidically to two or more bioreactors or vice versa.

11. The method as claimed in claim 1, wherein the strain used is a strain of Trichoderma reesei or of Trichoderma reesei modified by selective mutation or genetic recombination.

12. The method as claimed in claim 1, wherein the enzymes are cellulolytic or hemicellulolytic enzymes.

13. A plant for producing enzymes by a strain belonging to a filamentous fungus, characterized in that said plant comprises:—a stirred and aerated bioreactor operating in batch phase for performing a step of growing the fungi, in the presence of at least one carbon-based growth substrate;—a bubble column operating in fed-batch phase for performing a step of producing enzymes from the culture medium from the bioreactor;—means (13) of fluidic connection connecting the bioreactor to the bubble column for performing the transfer of the growth medium from the bioreactor to the bubble column;—means for injecting the dilution liquid (2;9;14) into the bioreactor and/or into the bubble column and/or into the means (13) of fluidic connection.

14. The plant as claimed in claim 13, wherein the bubble column has an internal volume at least two times greater than the internal volume of the stirred bioreactor.

15. The plant as claimed in claim 14, which comprises n stirred and aerated bioreactors and m bubble columns, where n is less than or equal to m.
